(19) United States
(12) Patent Application Publication
(10) Pub. No.: US 2026/0021204 A1
(43) Pub. Date: Jan. 22, 2026

(54) ANTI-SARS-COV-1 AND ANTI-SARS-COV-2 ACTIVATABLE RNASE GUIDE SEQUENCES

(71) Applicants: GEORGIA TECH RESEARCH CORPORATION, Atlanta, GA (US); EMORY UNIVERSITY, Atlanta, GA (US)

(72) Inventors: Daryll VANOVER, Duluth, GA (US); Philip J. SANTANGELO, Smyrna, GA (US); Chiara ZURLA, Atlanta, GA (US)

(21) Appl. No.: 18/994,913

(22) PCT Filed: Jul. 14, 2023
(86) PCT No.: PCT/US2023/070265

§ 371 Date: Jan. 15, 2025

Related U.S. Application Data
(60) Provisional application No. 63/389,647, filed on Jul. 15, 2022.

(51) Int. Cl. A61K 48/00; C12N 9/22; C12N 15/11

(52) U.S. Cl. CPC A61K 48/0058; C12N 9/226; C12N 15/11; C12N 2310/20

(57) ABSTRACT

The present disclosure relates to anti-SARS-COV-1 and anti-SARS-COV-2 activatable RNase guide sequences and methods of use for screening, treating, and/or preventing SARS infections and/or COVID-related diseases.

### CROSS-REFERENCE TO RELATED APPLICATIONS

This application is a U.S. National Stage application filed under 35 U.S.C. § 371 of PCT/US2023/070265, filed Jul. 14, 2023, which claims the benefit of U.S. Provisional Application No. 63/389,647, filed Jul. 15, 2022, each of which is expressly incorporated herein by reference in their entirety.

### STATEMENT REGARDING FEDERALLY SPONSORED RESEARCH

This invention was made with government support under HR0011-19-2-0008 awarded by the Defense Advanced Research Projects Agency. The government has certain rights in the invention.

### REFERENCE TO SEQUENCE LISTING

The sequence listing submitted on Jul. 14, 2023, as an .XML file entitled “10034-295WO1_2023_07_14_ST26” created on Jul. 14, 2023, and having a file size of 12,651 bytes is hereby incorporated by reference pursuant to 37 C.F.R. § 1.52 (e) (5).

### FIELD

The present disclosure relates to anti-SARS-COV-1 and anti-SARS-COV-2 activatable RNase guide sequences and methods of use for screening, treating, and/or preventing SARS infections and/or COVID-related diseases.

### BACKGROUND

Severe acute respiratory syndrome (SARS) is a viral respiratory disease caused by a SARS-associated coronavirus (SARS-COV-1). According to the Centers for Disease Control and Prevention (CDC), “SARS was first reported in Asia in February 2003. The illness spread to more than two dozen countries in North America, South America, Europe, and Asia before the SARS global outbreak of 2003 was contained.”

“The coronavirus SARS-COV-2 at the origin of COVID-19 shares more than 70% genetic similarity with SARS-COV-1 that was at the origin of 2003 SARS. Infection-associated symptoms are remarkably similar between SARS and COVID-19 diseases and are the same as community-acquired pneumonia symptoms. Antibiotics were empirically given to SARS patients in the early stages of the pathology whereas a different strategy has been decided in the management of COVID-19 pandemic with a worldwide shutdown.”

Furthermore, viruses such as SARS-COV-1 and SARS-COV-2 continuously evolve as changes in the genetic code (genetic mutations) occur during replication of the genome. As a result of these changes, it is difficult to treat or prevent SARS or COVID or to screen for patients that are infected by new strains of SARS or COVID.

Given the limitations described above, there is a need to develop new approaches for screening, treating, and/or preventing SARS infections and COVID-related diseases.

### SUMMARY

The present disclosure provides compositions and methods for screening, treating, and/or preventing SARS infections and/or COVID-related diseases.

In one aspect, disclosed herein is a synthetic guide RNA selected from the group consisting of a guide RNA comprising SEQ ID NO: 5 or SEQ ID NO: 6 joined to a second nucleotide sequence that is complementary to SARS-COV-1 and SARS-COV-2, a guide RNA comprising SEQ ID NO: 1, SEQ ID NO: 2, SEQ ID NO: 3, or SEQ ID NO: 4, a guide RNA comprising about 40-90 nucleotides in length and comprising at least 80% sequence identity to SEQ ID NO: 1, SEQ ID NO: 2, SEQ ID NO: 3, or SEQ ID NO: 4, and a guide RNA molecule comprising at least 50 nucleotides in length but no more than about 100 nucleotides in length and comprises SEQ ID NO: 1, SEQ ID NO: 2, SEQ ID NO: 3, or SEQ ID NO: 4.

In some embodiments, the second nucleotide sequence is about 20-30 nucleotides in length. In some embodiments, at least one nucleotide comprises a chemical 2′-modification. In some embodiments, at least one linkage between the nucleotides of the guide RNA comprises a modification. In some embodiments, at least one of base of the guide RNA comprises a modification.

In some embodiments, the guide RNA is mammalian RNA. In some embodiments, the guide RNA is human RNA.

In some embodiments, the guide RNA binds to a nucleotide sequence of a nucleocapsid of a SARS-COV-1 and SARS-COV-2 virus. In some embodiments, the guide RNA binds to a nucleotide sequence of a replicase of a SARS-COV-1 and SARS-COV-2 virus. In some embodiments, the nucleotide sequence comprises RNA.

In one aspect, disclosed herein is a pharmaceutical composition comprising the synthetic guide RNA of any preceding aspect and a pharmaceutically acceptable carrier. In some embodiments, the method further comprises a Cas13 molecule. In some embodiments, the Cas13 comprises a Cas13a. In some embodiments, the Cas13 molecule comprises a Cas13 protein or a Cas13 mRNA. In some embodiments, the Cas13 mRNA is active in the cytosol or in the nucleus of a cell. In some embodiments, the Cas13 molecule is derived from Leptotrichia buccalis, Leptotrichia wadeii, Prevotella sp. P5-125, Ruminoccocus flavefa-ciens XPD3002, Porphyromonas gulae, Ruminococcus flavefaciens, or Leptotrichia shahii.

In some embodiments, the pharmaceutically acceptable carrier comprises a lipid nanoparticle, a PBAE, or a PBAE-derived nanoparticle. In some embodiments, the composition is formulated to deliver the guide RNA molecule and/or Cas13 molecule to the cytosol or the nucleus of the cell.

In one aspect, disclosed herein is a kit comprising the synthetic guide RNA of any preceding aspect and a pharmaceutically acceptable carrier, or the pharmaceutical composition of any preceding aspect and a pharmaceutically acceptable carrier.

In some embodiments, the kit further comprises one or more buffered solutions selected from nuclease-free water, phosphate buffered saline, Tris-buffered saline, or saline.

In one aspect, disclosed herein is a method of treating or preventing a SARS-COV-1 or a SARS-COV-2 infection in a subject, the method comprising administering a therapeutically effective amount of at least one synthetic guide RNAs of any preceding aspect, a Cas13, or the pharmaceutical composition of any preceding aspect to said subject, wherein the method decreases a SARS-COV-1 or a SARS-COV-2 viral load in the subject relative to an untreated subject with a SARS-COV-1 or a SARS-COV-2 infection.

In one aspect, disclosed herein is a method of detecting a SARS-COV-1 or a SARS-COV-2 virus in a subject, the method comprising isolating a sample from the subject, and contacting the sample with a composition comprising the synthetic guide RNA of any preceding aspect, a Cas13, and a reporter gene, or a pharmaceutical composition of any preceding aspect and a reporter gene, wherein the composition detects a presence of the SARS-COV-1 or SARS-COV-2 virus.

In some embodiments, the method further comprises administering two or more different synthetic guide RNAs. In some embodiments, the two or more different synthetic guide RNAs comprises at least 80% sequence identity to SEQ ID NO: 1, SEQ ID NO: 2, SEQ ID NO: 3, or SEQ ID NO: 4. In some embodiments, the guide RNA is administered before or after the Cas13 molecule. In some embodiments, the guide RNA is administered alone or in combination with the Cas13.

In some embodiments, the method comprises administering the guide RNA in a delivery vehicle suitable for delivery to the cytosol or nucleus of a cell. In some embodiments, the method comprises administering the Cas13 in a delivery vehicle suitable for delivery to the cytosol or the nucleus of the cell. In some embodiments, the delivery vehicle comprises a lipid nanoparticle, a PBAE, or PBAE derived nanoparticle.

In some embodiments, the subject is a mammal. In some embodiments, the mammal is a human.

### BRIEF DESCRIPTION OF FIGURES

The accompanying figures, which are incorporated in and constitute a part of this specification, illustrate several aspects described below.

FIG. 1 shows a schematic of Cas13a-mediated degradation of viral RNA.

FIG. 2 show a schematic of the drugs of the disclosure.

FIG. 3 shows a schematic of how to generate anti-SARS-COV-1 and anti-SARS-COV-2 activatable RNase guide sequences.

FIGS. 4A, 4B, 4C, and 4D show Cas13 mediated knock-down of cytopathic effect upon SARS-COV-2 infection.

FIG. 5 shows a schematic of a nose cone nebulization system and the luminescent imaging assay.

FIG. 6 shows the results of the studies assessing the efficacy of mRNA doses and formulations as assessed by luminescent imaging.

FIG. 7 shows a hamster nebulizer setup with custom 3D printed nose cones sized for Syrian hamsters.

FIGS. 8A, 8B, and 8C show the radiance measurements assessing the delivery of Cas13-2A-NanoLuc with and without a guide RNA via delivery by nebulizer.

FIGS. 9A, 9B, and 9C show Cas13a mediated mitigation of SARS-COV-2 in vivo measured as a change in weight and viral load as a function of time post infection.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

By “reduce” or other forms of the word, such as “reducing” or “reduction,” means lowering of an event or characteristic (e.g., viral infection). It is understood that this is typically in relation to some standard or expected value, in other words it is relative, but that it is not always necessary for the standard or relative value to be referred to. For example, “reduces viral loads” means reducing the rate of viral growth or infection relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “detect” or “detecting” refers to an output signal released for the purpose of sensing of physical phenomenon. An event or change in environment is sensed and signal output released in the form of light.

The terms “treat,” “treating,” and grammatical variations thereof as used herein, include partially or completely delaying, alleviating, mitigating, or reducing the intensity of one or more attendant symptoms of a disorder or condition and/or alleviating, mitigating, or impeding one or more causes of a disorder or condition. Treatments according to the disclosure may be applied preventively, prophylactically, palliatively, or remedially. Treatments are administered to a subject prior to onset (e.g., before obvious signs of infection), during early onset (e.g., upon initial signs and symptoms of SARS infection), or after an established development of COVID.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

A “therapeutic composition” refers to at least one substance, molecule, or compound suitable for administering to a subject, wherein the composition further includes a pharmaceutical carrier. A non-limiting example includes a therapeutic composition comprises a nucleobase-poly-amino acid carrier and a sterile water-based solution.

An “effective amount” is an amount sufficient to affect beneficial or desired results. An effective amount can be administered in one or more administrations, applications, or dosages. “Effective amount” encompasses, without limitation, an amount that can ameliorate, reverse, mitigate, prevent, or diagnose a symptom or sign of a medical condition or disorder. Unless dictated otherwise, explicitly or by context, an “effective amount” is not limited to a minimal amount sufficient to ameliorate a condition. The severity of a disease or disorder, as well as the ability of a treatment to prevent, treat, or mitigate, the disease or disorder can be measured, without implying any limitation, by a biomarker or by a clinical parameter.

As used herein, the term infection refers to the invasion of tissues by pathogens, their multiplication, and reaction of host tissues to the infectious agent and any toxins they release. Infections can be caused by a wide range of pathogen, most common are viruses.

A “virus” is a microscopic infectious agent that replicates only inside the living cells of an organism. Viruses can infect all life forms, including mammalian and non-mammalian animals, plants, and other microorganisms. A complete virus, also known as a virion, consists of nucleic acid genetic material surrounded by a protective coat of protein called a capsid. Virus can have a lipid envelope derived from the infected host cell membrane. In general, there are five morphological virus types including helical, icosahedral, prolate, enveloped, and complex virus. A virus can either have a DNA or RNA genome, though a vast majority have RNA genomes. Irrespective of the type of nucleic acid genome, a viral genome can be either a single-stranded genome or a double-stranded genome.

A “protein”, “polypeptide”, or “peptide” each refer to a polymer of amino acids and does not imply a specific length of a polymer of amino acids. Thus, for example, the terms peptide, oligopeptide, protein, antibody, and enzyme are included within the definition of polypeptide. Reference also is made herein to peptides, polypeptides, proteins, and compositions comprising peptides, polypeptides, and proteins. As used herein, a polypeptide and/or protein is defined as a polymer of amino acids, typically of length ≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110). The term “amino acid,” includes but is not limited to amino acids contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods consider conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length may be used to describe a length over which percentage identity may be measured.

A “nucleotide” is a compound consisting of a nucleoside, which consists of a nitrogenous base and a 5-carbon sugar, linked to a phosphate group forming the basic structural unit of nucleic acids, such as DNA or RNA. The four types of nucleotides are adenine (A), cytosine (C), guanine (G), and thymine (T), each of which are bound together by a phosphodiester bond to form a nucleic acid molecule.

A “nucleic acid” is a chemical compound that serves as the primary information-carrying molecules in cells and make up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Percent identity may be measured over the length of an entire defined polynucleotide sequence or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length may be used to describe a length over which percentage identity may be measured.

A “full length” polynucleotide sequence is one containing at least a translation initiation codon (e.g., methionine) followed by an open reading frame and a translation termination codon. A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide.

“Complement” or “complementary” as used herein means Watson-Crick (e.g., A-T/U and CG) or Hoogsteen base pairing between nucleotides or nucleotide analogs of nucleic acid molecules.

The term “CRISPR/CAS,” “clustered regularly interspaced short palindromic repeats system,” or “CRISPR” refers to DNA loci containing short repetitions of base sequences. Each repetition is followed by short segments of spacer DNA from previous exposures to a virus. Bacteria and archaea have evolved adaptive immune defenses termed CRISPR-CRISPR-associated (Cas) systems that use short RNA to direct degradation of foreign nucleic acids. In bacteria, the CRISPR system provides acquired immunity against invading foreign DNA via RNA-guided DNA cleavage.

As used herein, “PBAE” or “PBAE-derived” polymers refer to polymers that respectively include a beta amino ester component or that were synthesized from or using a beta amino ester component.

Guide RNA Compositions and/or Kits

Provided here are illustrative embodiments of the disclosed technology. These embodiments are illustrative only and do not limit the scope of the present disclosure or of the claims attached.

The present disclosure provides for synthetic anti-SARS-COV-1 and anti-SARS-COV-2 guide RNA used to target or guide a Cas13 to SARS-COV-1 and SARS-COV-2. The guide RNAs are used to detect, screen, treat, and/or prevent a SARS-COV-1 and SARS-COV-2 infection.

The individual synthetic guide RNAs have two portions: a portion that interacts with the CRISPR Cas molecule; and the portion that targets the molecule to the SARS-COV-1 or SARS-CoV-2 genomic or messenger RNA (mRNA) sequences. In some embodiments, the guide RNA includes modified nucleotides and/or modifications in the linkages between the bases in the guide RNA.

In some embodiments, the guide RNA comprises SEQ ID NO: 1, SEQ ID NO: 2, SEQ ID NO: 3, or SEQ ID NO: 4 as shown in Table 1. The direct repeat region of the guide RNA sequences are bolded. All guide RNAs are given in 5′→3′ format.

In one aspect, disclosed herein is a synthetic guide RNA selected from the group consisting of a) a guide RNA comprising SEQ ID NO: 5 or SEQ ID NO: 6 joined to a second nucleotide sequence that is complementary to SARS-COV-1 and SARS-COV-2, b) a guide RNA comprising SEQ ID NO: 1, SEQ ID NO: 2, SEQ ID NO: 3, or SEQ ID NO: 4, c) a guide RNA comprising about 40-90 nucleotides in length and comprising at least 80% sequence identity to SEQ ID NO: 1, SEQ ID NO: 2, SEQ ID NO: 3, or SEQ ID NO: 4, and d) a guide RNA molecule comprising at least 50 nucleotides in length but no more than about 100 nucleotides in length and comprises SEQ ID NO: 1, SEQ ID NO: 2, SEQ ID NO: 3, or SEQ ID NO: 4.

In some embodiments, the guide RNA of step c) comprises about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 nucleotides in length. In some embodiments, the guide RNA may be about 40-90, alternatively about 60-80, alternatively about 55-75, alternatively about 50-80, alternatively about 50-70, alternatively about 65-85, alternatively about 50, alternatively about 55, alternatively about 58, alternatively about 60, alternatively about 65, alternatively about 70, alternatively about 75, alternatively about 80, alternatively about 85, alternatively about 90 nucleotides in length. In some embodiments, the guide RNA molecule of step d) comprising 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nucleotides in length. In some embodiments, the guide RNA comprises at least 50, alternatively at least about 55, alternatively at least about 60, alternatively at least about 65, alternatively at least 70, alternatively at least about 75, alternatively at least about 80, alternatively at least about 85, alternatively at least about 90 nucleotides, but no more than about 100 nucleotides in length.

In some embodiments, the guide RNA of step c) comprises a nucleotide sequence comprising at least 80% identity to SEQ ID NO: 1, SEQ ID NO: 2, SEQ ID NO: 3, or SEQ ID NO: 4. In some embodiments, the guide RNA molecule of step c) comprises 80%, 81%, 82%, 83%, 84%, 85%, 86%, 87%, 88%, 89%, 90%, 91%, 92%, 93%, 94%, 95%, 96%, 97%, 98%, or 99% identity of SEQ ID NO: 1. In some embodiments, the guide RNA molecule of step c) comprises 80%, 81%, 82%, 83%, 84%, 85%, 86%, 87%, 88%, 89%, 90%, 91%, 92%, 93%, 94%, 95%, 96%, 97%, 98%, or 99% identity of SEQ ID NO: 2. In some embodiments, the guide RNA molecule of step c) comprises 80%, 81%, 82%, 83%, 84%, 85%, 86%, 87%, 88%, 89%, 90%, 91%, 92%, 93%, 94%, 95%, 96%, 97%, 98%, or 99% identity of SEQ ID NO: 3. In some embodiments, the guide RNA molecule of step c) comprises 80%, 81%, 82%, 83%, 84%, 85%, 86%, 87%, 88%, 89%, 90%, 91%, 92%, 93%, 94%, 95%, 96%, 97%, 98%, or 99% identity of SEQ ID NO: 4.

In some embodiments, the second nucleotide sequence is about 20-30 nucleotides in length. In some embodiments, the second nucleotide sequence comprises about 20, 21, 22, 23, 24, 25, 26, 27, 28, 29, 30 nucleotides in length. In some embodiments, the second nucleotide sequence comprises RNA. In some embodiments, the second nucleotide sequence comprises DNA.

In some embodiments, at least one nucleotide comprises a chemical 2′-modification. In some embodiments, at least one linkage between the nucleotides of the guide RNA comprises a modification. In some embodiments, at least one of base of the guide RNA comprises a modification.

In some embodiments, the chemical modification or modification is selected from 5-formylcytidine (5fC), 5-methylcytidine (5meC), 5-methoxycytidine (5moC), 5-hydroxycytidine (5hoC), 5-hydroxymethylcytidine (5hmC), 5-formyluridine (5fU), 5-methyluridine (5-meU), 5-methoxyuridine (5moU), 5-carboxymethylesteruridine (5camU), pseudouridine (Ψ), N1-methylpseudouridine (me1Ψ), N6-methyladenosine (me6A), or thienoguanosine (thG). In some embodiments, the chemical modification or modification is selected from 2′-O-methyl (2′-O-Me), 2′-Fluoro (2′-F), 2′-deoxy-2′-fluoro-beta-D-arabino-nucleic acid (2′F-ANA), 4′-S, 4′-SFANA, 2′-azido, UNA, 2′-O-methoxy-ethyl (2′-O-ME), 2′-O-Allyl, 2′-O-Ethylamine, 2′-O-Cyanoethyl, Locked nucleic acid (LAN), Methylene-cLAN, N-MeO-amino BNA, or N-MeO-aminooxy BNA.

Modified bases include, but are not limited to, those found in the following nucleoside analogs: pseudouridine, N1-methyl-pseudouridine, 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O (6)-methylguanine, and 2-thiocytidine. Modified sugars include, but are not limited to, 2′-fluororibose, ribose, 2′-deoxyribose, 3′-azido-2′,3′-dideoxyribose, 2′,3′-dideoxyribose, arabinose (the 2′-epimer of ribose), acyclic sugars, and hexoses. The nucleosides may be strung together by linkages other than the phosphodiester linkage found in naturally occurring DNA and RNA. Modified linkages include, but are not limited to, phosphorothioate and 5′-N-phosphoramidite linkages. Combinations of the various modifications may be used in a single guide RNA. These modified guide RNAs may be provided by any means known in the art; however, as will be appreciated by those of skill in this art, the modified guide RNAs are preferably prepared using synthetic chemistry in vitro.

In some embodiments, the linkage comprises a chemical modification or moiety selected from the group consisting of carbamate, ether, ester, amide, imine, amidine, aminotrizine, hydrozone, disulfide, thioether, thioester, phosphorothioate, phosphorodithioate, sulfonamide, sulfonate, fulfone, sulfoxide, urea, thiourea, hydrazide, oxime, triazole, photolabile linkage, C—C bond forming group such as Diels-Alder cyclo-addition pair or ring-closing metathesis pair, Michael reaction pair, Phosphorothioate (PS), Boranophosphate, phosphodithioate (PS2), 3′,5′-amide, N3′-phosphoramidate (NP), Phosphodiester (PO), or 2′,5′-phosphodiester (2′,5′-PO). In some embodiments, the linkages between the individual nucleotides is modified. The linkages include, but are not limited to phosphorothioate and/or phosphorodithioate linkages.

In some embodiments, the guide RNA comprises mammalian RNA. In some embodiments, the guide RNA comprises human RNA.

In some embodiments, the guide RNA binds to a nucleotide sequence of a nucleocapsid of a SARS-COV-1 and SARS-COV-2 virus. In some embodiments, the guide RNA binds to a nucleotide sequence of a replicase of a SARS-COV-1 and SARS-COV-2 virus. In some embodiments, the nucleotide sequence comprises RNA.

In one aspect, disclosed herein is a pharmaceutical composition comprising the synthetic guide RNA of any preceding aspect and a pharmaceutically acceptable carrier.

In some embodiments, the pharmaceutical composition further comprises a Cas13. In some embodiments, the Cas13 comprises a Cas13a. In some embodiments, the Cas13 comprises a Cas13 protein or a Cas13 mRNA. In some embodiments, the Cas13a comprises a Cas13a protein or a Cas13a mRNA. While in some embodiments, Cas13 mRNA is used, in other embodiments the Cas13 protein is used instead.

Cas13a, also known as C2C2, is a CRISPR-associated nuclease that cuts single-stranded and collateral RNA. It was originally discovered in Leptotrichia shahii (also referred to as LshCas13a) and is composed of two higher eukaryotes and prokaryotes nucleotide-binding (HEPN) domains that can distinguish between the CRISPR RNA and the targeted single-stranded RNA to edit. Since its discovery, additional isoforms of Cas13 have been discovered, namely Cas13b, Cas13c, Cas13d, Cas13X, and Cas13Y. Once Cas13 binds its target RNA the enzyme will exhibit non-specific RNase activity. As used throughout the present disclosure “Cas13” can refer to any Cas13 isoform, including, for example, Cas13a, Cas13b, Cas13c, Cas13d, Cas13X, or Cas13Y. Any known Cas13 mRNA may be used.

In some embodiments, the Cas13 mRNA is active in the cytosol or in the nucleus of a cell. In some embodiments, the Cas13 mRNA is active in the nucleus of a cell. In some embodiments, the Cas13 is derived from Leptotrichia buccalis, Leptotrichia wadeii, Prevotella sp. P5-125, Ruminoccocus flavefa-ciens XPD3002, Porphyromonas gulae, Ruminococcus flavefaciens, or Leptotrichia shahii.

In some embodiments, the pharmaceutically acceptable carrier comprises a lipid nanoparticle. In some embodiments, the pharmaceutically acceptable carrier comprises a PBAE or PBAE-derived nanoparticle. In some embodiments, the pharmaceutically acceptable carrier comprises a structure as shown in Compound 1, or derivatives thereof. In some embodiments, the PBAE or the PBAE-derived nanoparticle comprises Compound 1, or derivatives thereof.

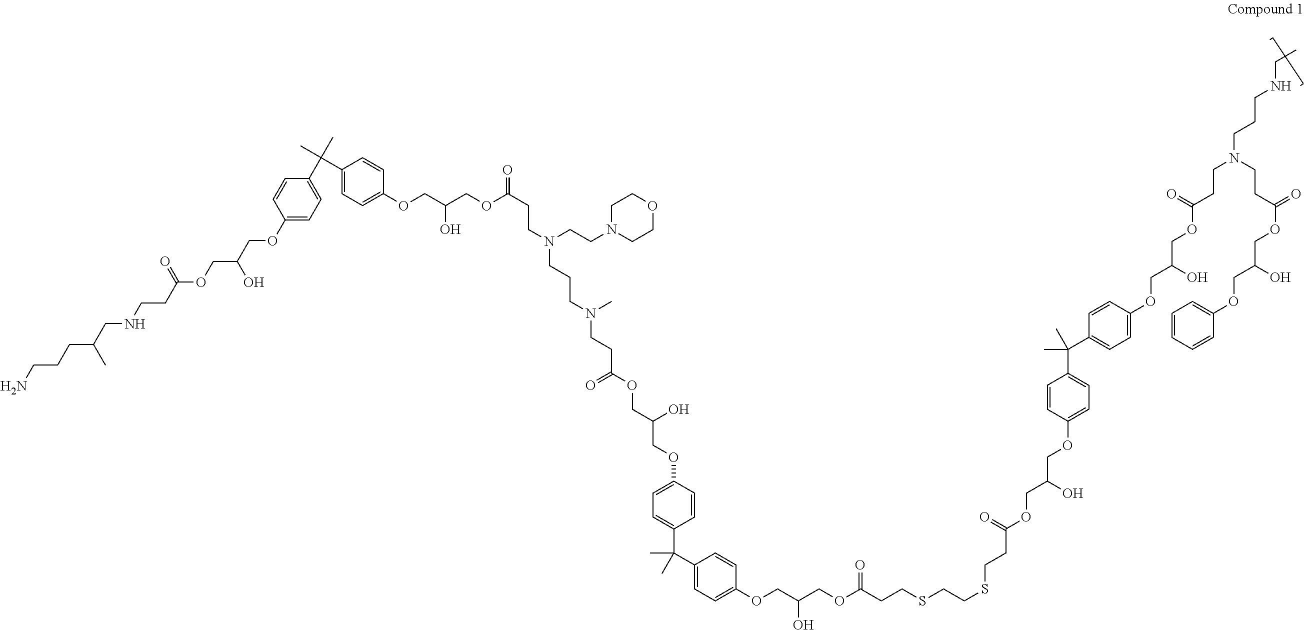

In some embodiments, the pharmaceutical composition is formulated to deliver the guide RNA molecule and/or Cas13 molecule to the cytosol of a cell. In some embodiments, the pharmaceutical composition is formulated to deliver the guide RNA molecule and/or Cas13 molecule to the nucleus of a cell.

In some embodiments, the pharmaceutically acceptable carrier comprises a suitable delivery vehicle for delivering RNA to a cell. The delivery vehicle for use in the constructs of the disclosure includes any delivery vehicle suitable for delivering an mRNA molecule to a cell. Examples of such delivery vehicles include lipid nanoparticles and PBAE (Poly(β-Amino Ester)) nanoparticles. In some embodiments, the delivery vehicle is formulated for pulmonary administration. In some embodiments, the delivery vehicle comprises a PBAE nanoparticle. In some embodiment, the delivery vehicle is formulated for systemic delivery.

Examples of suitable lipid nanoparticles are disclosed in Tenchov, R. et al. “Lipid Nanoparticles—From Liposomes to mRNA Vaccine Delivery, a Landscape of Research Diversity and Advancement,” ACS NANO 15 (11), 16982-17015 (2021) and the patent documents referenced therein, the disclosure of which is incorporated herein by reference as it pertains to lipid nanoparticles.

In some embodiments, the delivery vehicle comprises polymers containing beta-amino-ester and beta-thio-ester groups (denoted BAE/BTE-containing polymers). In some embodiments, the delivery vehicle comprises polymers containing beta-amino-ester and beta-thio-ester groups (denoted BAE/BTE-containing polymers) as disclosed in U.S. Provisional Patent Application No. 63/287,691, filed Dec. 9, 2021, the entire contents of which are incorporated herein by reference.

In some embodiments, the delivery vehicle comprises a lipid nanoparticle. In some embodiments, the delivery vehicle comprises both the guide RNA and the mRNA encoding Cas13 in the same delivery vehicle. In some embodiments, multiple different types of delivery vehicles are employed with one delivery vehicle containing the guide RNA and the other containing the mRNA encoding Cas13. In some embodiments, the compositions contain more than one type of guide RNA.

In one aspect, disclosed herein is a kit comprising the synthetic guide RNA of any preceding aspect and a pharmaceutically acceptable carrier, or the pharmaceutical composition of any preceding aspect and a pharmaceutically acceptable carrier.

In some embodiments, the kit further comprises one or more buffered solutions selected from nuclease-free water, phosphate buffered saline, Tris-buffered saline, or saline.

Methods of Detecting, Screening, Preventing, and/or Treating SARS

In one aspect, disclosed herein is a method of detecting a SARS-COV-1 or a SARS-COV-2 virus in a subject, the method comprising isolating a sample from the subject, and contacting the sample with a composition comprising the synthetic guide RNA of any preceding aspect, a Cas13, and a reporter gene, or a pharmaceutical composition of any preceding aspect and a reporter gene, wherein the composition detects a presence of the SARS-COV-1 or SARS-COV-2 virus.

In one aspect, disclosed herein is a method of treating or preventing a SARS-COV-1 or a SARS-COV-2 infection in a subject, the method comprising administering a therapeutically effective amount of at least one synthetic guide RNAs of any preceding aspect, a Cas13, or the pharmaceutical composition of any preceding aspect to said subject, wherein the method decreases a SARS-COV-1 or a SARS-COV-2 viral load in the subject relative to an untreated subject with a SARS-COV-1 or a SARS-COV-2 infection.

In one aspect disclosed herein is a method screening to detect the presence of a SARS-CoV-1 or a SARS-COV-2 virus in a sample. When used in the screening methods, the guide RNA is used together with Cas13 molecule and a reporter of Cas13 activity. The screening methods involve contacting the sample with the guide RNA and Cas13 molecule and then testing for the activity of the Cas13 reporter. If activity is detected, then the sample is positive for SARS-COV-1, SARS-COV-2, and/or COVID.

In some embodiments, the method further comprises administering two or more different synthetic guide RNAs. In some embodiments, the two or more different synthetic guide RNAs comprises at least 80% sequence identity to SEQ ID NO: 1, SEQ ID NO: 2, SEQ ID NO: 3, or SEQ ID NO: 4.

In some embodiments, the guide RNA is administered before or after the Cas13. In some embodiments, the guide RNA is administered alone or in combination with the Cas13.

In some embodiments, the method comprises more than one type of Cas molecule. In some embodiments, the method comprises more than one type of Cas mRNA. In some embodiments, the method comprises more than one type of Cas protein. In some embodiments, the method comprises administering the guide RNA in a delivery vehicle suitable for delivery to the cytosol or nucleus of a cell. In some embodiments, the method comprises administering the Cas13 in a delivery vehicle suitable for delivery to the cytosol or the nucleus of the cell. In some embodiments, the delivery vehicle comprises a lipid nanoparticle, a PBAE, or a PBAE-derived nanoparticle.

In some embodiments, the reporter gene comprises luciferase, green fluorescent protein (GFP), yellow fluorescent protein (YFP), blue fluorescent protein (BFP), cyane fluorescent protein (CFP), monomeric red fluorescent protein (mRFP), Discosoma striata (DsRed), mCherry, mOrange, tdTomato, mSTrawberry, mPlum, photoactivatable GFP (PA-GFP), Venus, Kaede, monomeric kusabira orange (mKO), Dronpa, enhanced CFP (ECFP), Emerald, Cyan fluorescent protein for energy transfer (CyPet), super CFP (SCFP), Cerulean, photoswitchable CFP (PS-CFP2), photoactivatable RFP1 (PA-RFP1), photoactivatable mCherry (PA-mCherry), monomeric teal fluorescent protein (mTFP1), Eos fluorescent protein (EosFP), Dendra, TagBFP, TagRFP, enhanced YFP (EYFP), Topaz, Citrine, yellow fluorescent protein for energy transfer (YPet), super YFP (SYFP), enhanced GFP (EGFP), Superfolder GFP, T-Sapphire, Fucci, mKO2, mOrange2, mApple, Sirius, Azurite, EBFP, and/or EBFP2.

In some embodiments, the presence of a SARS-COV-1 or a SARS-COV-2 virus is detected using methods including, but not limited to polymerase chain reactions (PCR) (including, but not limited to PCR, RT-PCR (real-time PCR) or RT-PCR (reverse transcribed PCR)), sequencing (including, but not limited to RNA sequencing (RNA-seq) and next generation sequencing (NGS)), western blot, direct ELISA, indirect ELISA, high-performance liquid chromatography (HPLC), liquid chromatography-mass spectrometry, mass spectrometry, immunoprecipitation, and immunostaining.

In some embodiments, the sample is derived from blood, saliva, urine, tissue, mucous, or other bodily tissues or fluids.

The synthetic guide RNA or the pharmaceutical composition may be administered in such amounts, time, and route deemed necessary in order to achieve the desired result. The exact amount of the synthetic guide RNA or the pharmaceutical composition will vary from subject to subject, depending on the species, age, and general condition of the subject, the severity of the infection, the particular synthetic guide RNA or the pharmaceutical composition, its mode of administration, its mode of activity, and the like. The synthetic guide RNA or the pharmaceutical composition is preferably formulated in dosage unit form for ease of administration and uniformity of dosage. It will be understood, however, that the total daily usage of the synthetic guide RNA or the pharmaceutical composition will be decided by the attending physician within the scope of sound medical judgment. The specific therapeutically effective dose level for any particular subject will depend upon a variety of factors including the viral infection being treated and the severity of the infection; the activity of the synthetic guide RNA or the pharmaceutical composition employed; the specific synthetic guide RNA or the pharmaceutical composition employed; the age, body weight, general health, sex and diet of the patient; the time of administration, route of administration, and rate of excretion of the specific synthetic guide RNA or the pharmaceutical composition employed; the duration of the treatment; drugs used in combination or coincidental with the specific synthetic guide RNA or the pharmaceutical composition employed; and like factors well known in the medical arts.

The synthetic guide RNA or the pharmaceutical composition may be administered by any route. In some embodiments, the synthetic guide RNA or the pharmaceutical composition is administered via a variety of routes, including mucosal, nasal, buccal, oral, by intratracheal instillation, bronchial instillation, inhalation, as an oral spray, nasal spray, aerosol, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), enteral, and/or sublingual. In general, the most appropriate route of administration will depend upon a variety of factors including the nature of the synthetic guide RNA or the pharmaceutical composition (e.g., its stability in the environment of the respiratory system), the condition of the subject (e.g., whether the subject is able to tolerate nasal administration), etc.

The exact amount of synthetic guide RNA or the pharmaceutical composition required to achieve a therapeutically or prophylactically effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

The concentration of active agent(s) can vary widely and will be selected primarily based on activity of the active ingredient(s), body weight and the like in accordance with the particular mode of administration selected and the patient's needs. Concentrations, however, will typically be selected to provide dosages ranging from about 0.1 or 1 mg/kg/day to about 50 mg/kg/day and sometimes higher. Typical dosages range from about 3 mg/kg/day to about 3.5 mg/kg/day, preferably from about 3.5 mg/kg/day to about 7.2 mg/kg/day, more preferably from about 7.2 mg/kg/day to about 11.0 mg/kg/day, and most preferably from about 11.0 mg/kg/day to about 15.0 mg/kg/day. In certain preferred embodiments, dosages range from about 10 mg/kg/day to about 50 mg/kg/day. In certain embodiments, dosages range from about 20 mg to about 50 mg given orally twice daily. It will be appreciated that such dosages may be varied to optimize a therapeutic and/or prophylactic regimen in a particular subject or group of subjects.

In some embodiments, the synthetic guide RNA or the pharmaceutical composition is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more times. In some embodiments, synthetic guide RNA or the pharmaceutical composition is administered daily. In some embodiments, synthetic guide RNA or the pharmaceutical composition is administered every day, every 2 days, every 3 days, every 4 days, every 5 days, every 6 days, every 7 days, or more. In some embodiments, synthetic guide RNA or the pharmaceutical composition is administered every week, every 2 weeks, every 3 weeks, every 4 weeks, or more. In some embodiments, synthetic guide RNA or the pharmaceutical composition is administered every month, every 2 months, every 3 months, every 4 months, every 5 months, every 6 months, every 7 months, every 8 months, every 9 months, every 10 months, every 11 months, every 12 months, or more.

In one aspect, disclosed herein is synthetic guide RNA or the pharmaceutical composition of any preceding aspect and a pharmaceutically acceptable carrier selected from an excipient, a diluent, a salt, a buffer, a stabilizer, a lipid, an emulsion, a nanoparticle, and a cream. One or more active agents (e.g. the synthetic guide RNA) can be administered in the “native” form or, if desired in the form of salts, esters, amides, prodrugs, or a derivative that is pharmacologically suitable. Salts, esters, amides, prodrugs, and other derivatives of the active agents can be prepared using standards procedures known to those skilled in the art of synthetic organic chemistry and described, for example, by March (1992) Advanced Organic Chemistry; Reactions, Mechanisms, and Structure, 4^{th} Ed. N.Y. Wiley-Interscience.

In some embodiments, the subject is a mammal. In some embodiments, the mammal is a human.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Cas13a-Mediated Degradation of Viral RNA

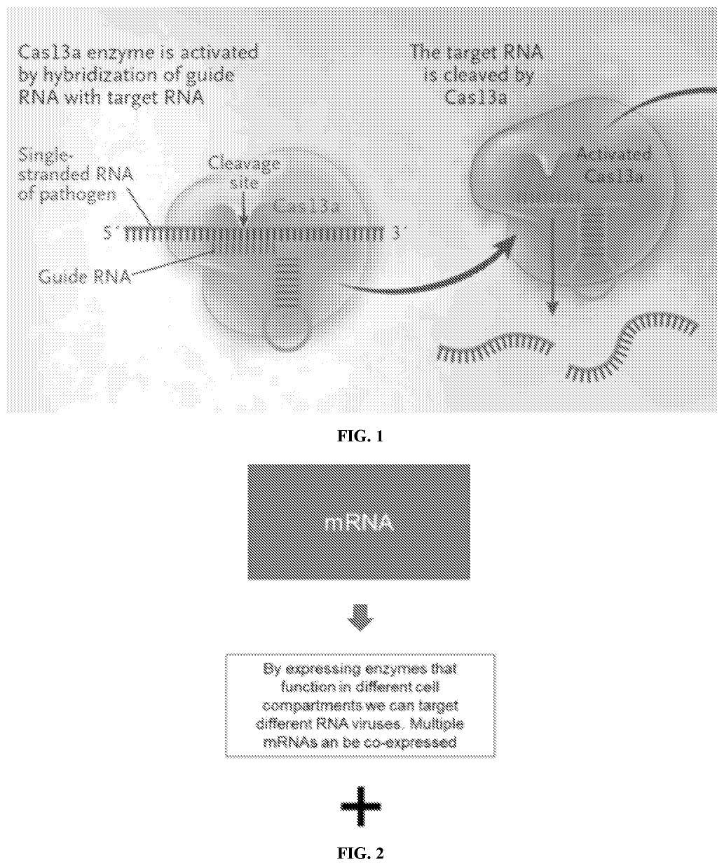

FIG. 1 shows a schematic of Cas13a-mediated degradation of viral RNA. Cas13a enzyme is synthesized in cells upon delivery of the mRNA that encodes for it, along with the guide RNA. The Cas13a-encoding mRNA may be synthesized in the lab while the guide RNA may be purchased from different companies (such as, for example, IDT and Synthego). The system employs two types of Cas13a mRNAs: one that expresses an enzyme that functions in the cell cytosol and one that expresses an enzyme that functions in the nucleus. The two mRNAs can be co-delivered, to have enzymes that function in both cell compartments. This is relevant because some, but not all, viruses replicate only in the cytosol (e.g. SARS-COV-2).

### Example 2: Schematic Arrangement of the Constructs

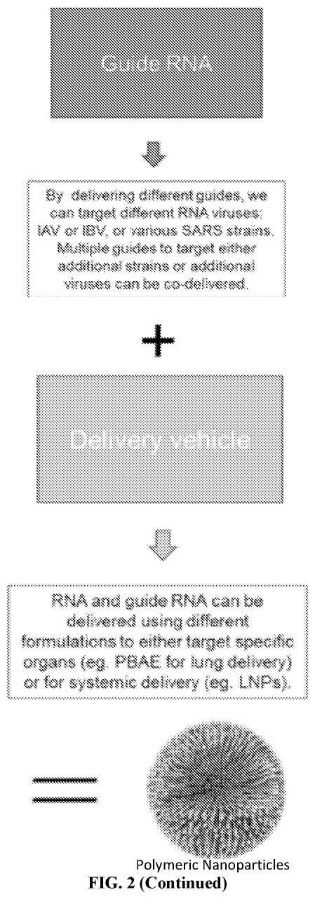

As noted above, the constructs disclosed herein treat SARS or COVID. In certain embodiments, the drug comprises at least one of three components: (1) mRNA encoding Cas13; (2) a guide RNA; and (3) a delivery vehicle. A schematic of such a construct is shown in FIG. 2.

### Example 3: Anti-SARS-COV-1 and Anti-SARS-COV-2 Activatable RNase Guide Sequences

Using the general procedure shown in FIG. 3, specific anti-SARS-COV-1 and anti-SARS-COV-2 activatable RNase guide sequences were designed. All of the guide sequence have a direct repeat region on the 5′ end of the guide sequence that is highly similar to:

(SEQ ID NO: 5)
5′-GGACCACCCCAAAAAUGAAGGGGACUAAAAC-3′

Alternatively, the direct repeat region on the 5′ end of the guide sequence is highly similar to:

(SEQ ID NO: 6)
5′-GGAUUUAGACCACCCCAAAAAUGAAGGGGACUAAAACA-3′.

This is followed by the region of the guide that “targets” the strand of RNA to be cleaved. These sequences are shown in Table 1 below. With reference to Table 1, the direct repeat region is bolded. All guide RNAs are given in 5′→3′ format.

TABLE 1
Guide mRNA sequences

| Guide | |
|---|---|
| Name | Full Guide Sequence |
| N3.1 | GGACCACCCCAAAAAUGAAGGGGACUAAAACGGUGUAU |
| | UCAAGGCUCCCUCAGUUGCA (SEQ ID NO: 1) |
| N3.2 | GGACCACCCCAAAAAUGAAGGGGACUAAAACGUAUUCAA |
| | GGCUCCCUCAGUUGCAACC (SEQ ID NO: 2) |
| N11.2 | GGACCACCCCAAAAAUGAAGGGGACUAAAACCUGUUGGU |
| | GGGAAUGUUUUGUAUGCGU (SEQ ID NO: 3) |
| R5.1 | GGACCACCCCAAAAAUGAAGGGGACUAAAACACGACAUC |
| | AGUACUAGUGCCUGUGCCG (SEQ ID NO: 4) |

The guides target highly conserved regions in the replicase and nucleocapsid regions of the genome. The nucleocapsid sites also allow Cas13a to target any of the sub genomic RNAs produced by the virus.

### Example 4: Cas13a Mediated Knock-Down of Cytopathic Effect Upon SARS-COV-2 Infection

Nine different guides were screened, by first transfecting Vero E6 cells overnight with the cytoplasmic version of Cas13a and guides, and then infecting with MOI=˜ 0.1 of SARS-COV-2. Note, only the cytoplasmic version was used, because the virus replicates in this cell compartment. Prophylactic delivery was used because this virus exhibits very rapid kinetics in Vero E6 cells (they are interferon deficient). At one hour post injection, an Avicel overlay was added to the wells and cytopathic effect (CPE) was evaluated at 60 hrs PI. The data was quantified by image analysis.

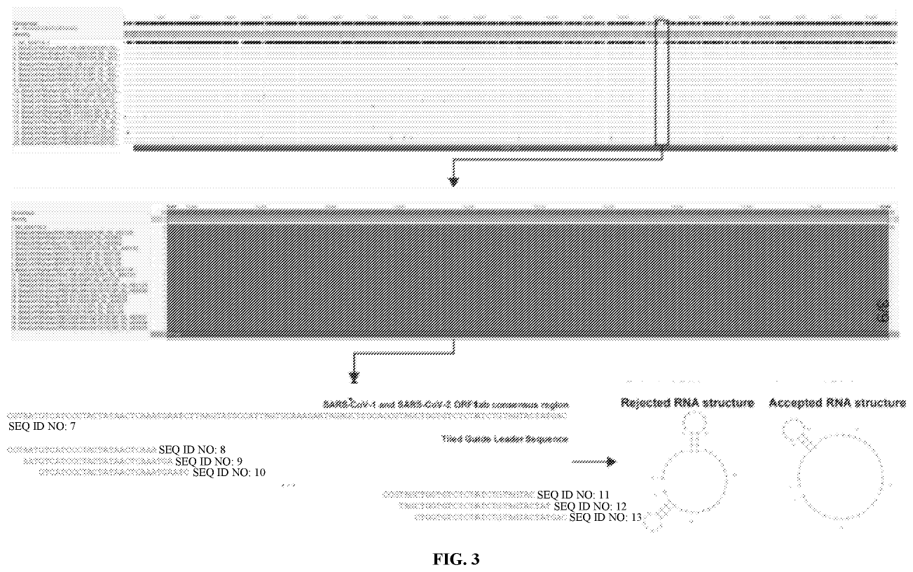

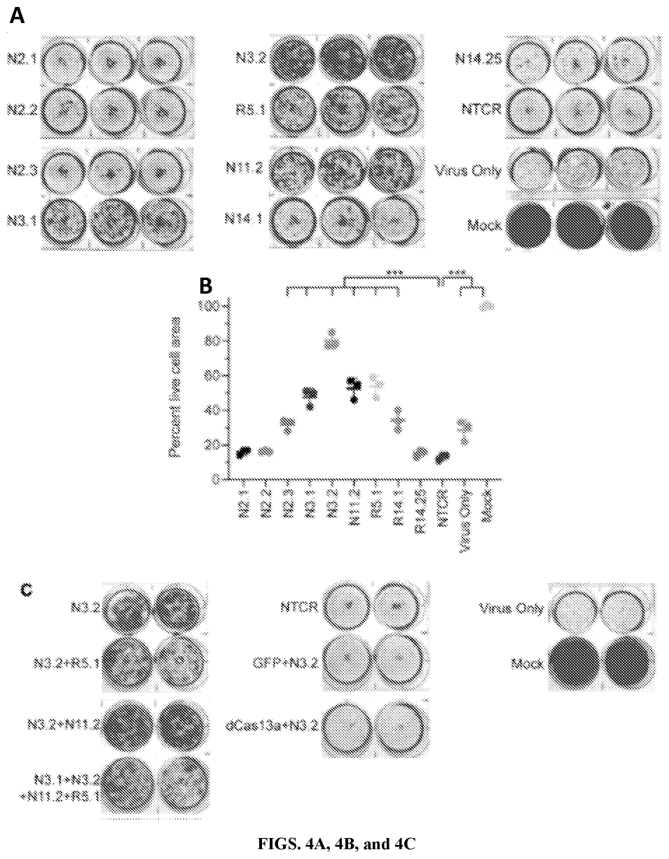

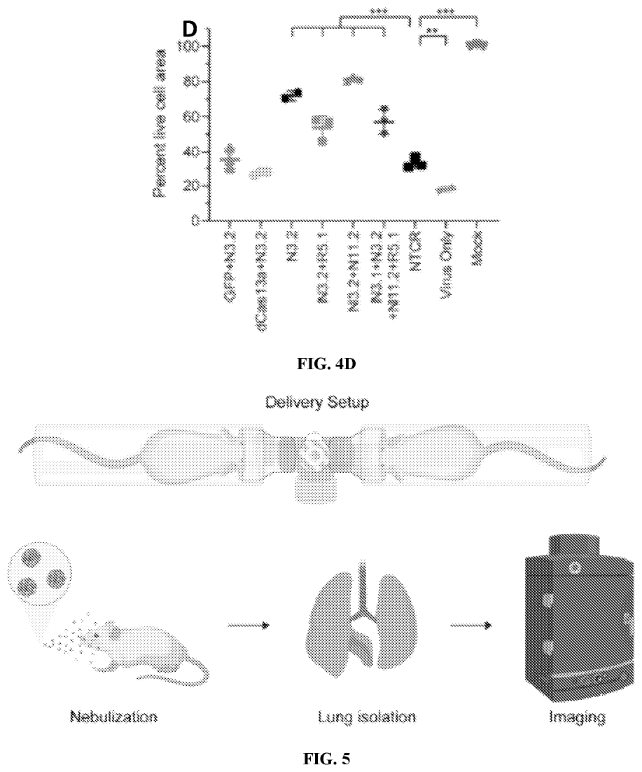

The results are shown in FIG. 4A. From this screen, guides N3.2 (nucleocapsid), N3.1 (nucleocapsid), R5.1 (replicase), and N11.2 (nucleocapsid) were found to have an impact on cytopathic effect (CPE), with guide 3.2 exhibiting the lowest CPE (see Table 1 above for sequences).

The experiment was repeated by first transfecting Vero E6 cells overnight with the cytoplasmic version of Cas13 and either individual or combinations of guides and including all controls (dCas 13 and GFP), and then infecting with MOI=˜ 0.1 of SARS-COV-2. At one hour post injection, an Avicel overlay was added to the wells and cytopathic effect (CPE) was evaluated at 60 hrs PI. The data was again quantified by image analysis. The results of this testing are shown in FIG. 4B. The combination of N3.2 and N11.2 over 72% reduction in cell death with over 80% of cells remaining in the plate.

### Example 5: Delivery of RNA Via Nose Cone Nebulization

A system to deliver mRNA to mice lungs via nose cone nebulization was constructed. FIG. 5A shows a nebulizer-based delivery system for mice. FIG. 5A additionally shows the workflow for assessing mRNA delivery in mice by 1) nebulizing a luminescent reporter mRNA, 2) isolating the lungs, and 3) assessing protein expression in the lungs via luminescent imaging.

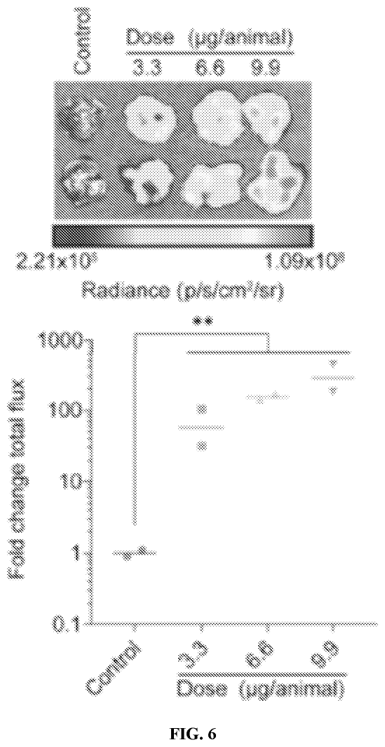

The efficacy of mRNA doses and formulations were assessed by luminescent imaging using a luminescent mRNA reporter construct. This approach is applicable to any delivery vehicle of interest. FIG. 6 shows the results of the studies assessing the efficacy of mRNA doses in BALB/c mice and formulations were assessed by luminescent imaging.

### Example 6: Nebulizer Based Delivery In Vivo in Hamsters

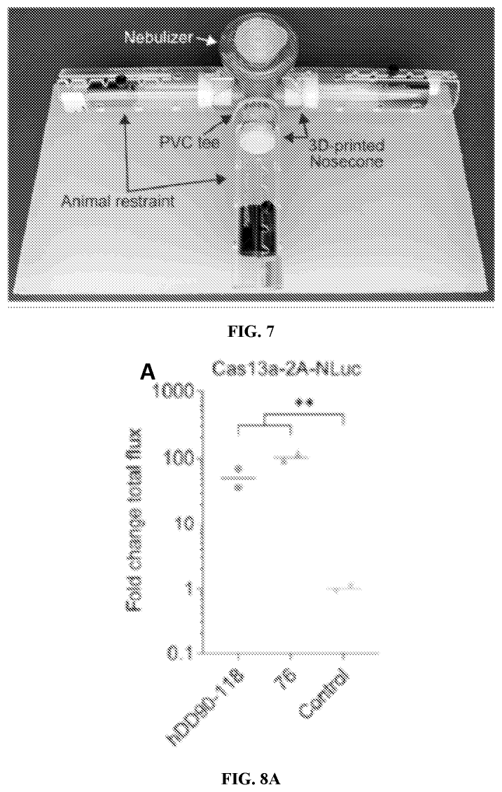

In addition to mice, the Cas13a mRNA delivery was assessed in hamsters. The hamster nebulizer setup with custom 3D printed nose cones sized for Syrian hamsters is shown in FIG. 7.

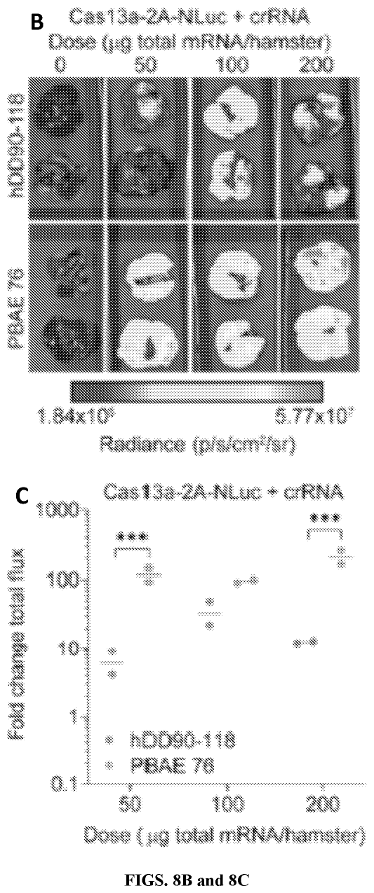

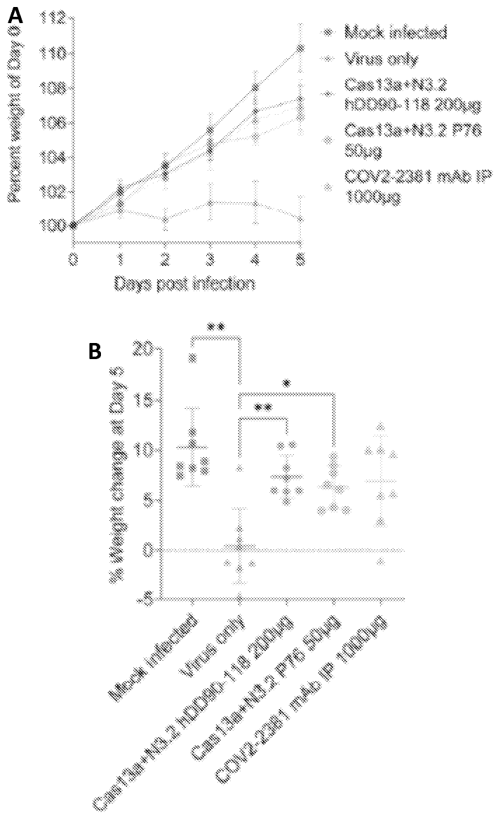

FIG. 8A shows radiance measurements assessing the delivery of Cas13-2A-NanoLuc via delivery by nebulizer by either hDD90-118 or PBAE 76 at 0.3 mg/kg. PBAE 76 has the structure shown in Compound 1, and is otherwise described in U.S. Provisional Patent Application No. 63/287,691, filed Dec. 9, 2021, incorporated herein by reference. Lungs were extracted and analyzed by luminescent imaging for protein expression. FIGS. 8B and 8C shows radiance measurements assessing the delivery of Cas13-2A-NanoLuc along with a guide RNA via delivery by nebulizer by either hDD90-118 or PBAE 76 at different doses. The results in FIG. 8C are a quantification of the data shown in FIG. 8B. Critically, PBAE 76 was more potent at delivering both the Cas13 mRNA and the guide RNA simultaneously.

### Example 7: Cas13a Mediated Mitigation of SARS-COV-2 In Vivo

Hamsters were dosed 24 hours prior to infection with indicated amounts of Cas13a mRNA with the N3.2 guide RNA using either hDD90-118 or PBAE 76. One group was treated with 10 mg/kg of anti-SARS-COV-2 COV2-2381 monoclonal antibody via intraperitoneal injection. The untreated, infected, and uninfected groups were not treated.

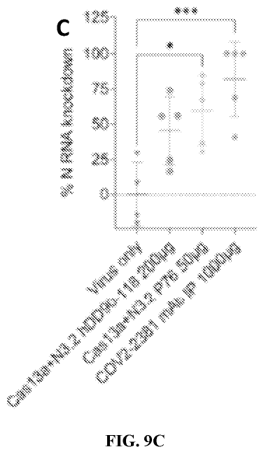

The Cas13 groups and untreated, infected group were infected on day 0 with 103 PFU of SARS-COV-2 WA-1 strain. Weights were tracked daily and normalized to day 0 weight as shown in FIG. 9A. The percentage weight change at day 5 is presented in FIG. 9B. Critically, the Cas13+N3.2 group delivered with PBAE76 at one-quarter the dose of the group delivered with hDD90-118 both had significantly higher weights at day 5 compared to the virus only group. The lungs from these animals at day 5 were analyzed for SARS-COV-2 N RNA via qPCR. The results are shown in FIG. 9C. Critically, only Cas13+N3.2 delivered with PBAE76 and the IP antibody groups displayed significant knockdown of the lung viral load.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

1. A synthetic guide RNA selected from the group consisting of:
a) a guide RNA comprising SEQ ID NO: 5 or SEQ ID NO: 6 joined to a second nucleotide sequence that is complementary to SARS-COV-1 and SARS-COV-2;
b) a guide RNA comprising SEQ ID NO: 1, SEQ ID NO: 2, SEQ ID NO: 3, or SEQ ID NO: 4;
c) a guide RNA comprising about 40-90 nucleotides in length and comprising at least 80% sequence identity to SEQ ID NO: 1, SEQ ID NO: 2, SEQ ID NO: 3, or SEQ ID NO: 4; and
d) a guide RNA molecule comprising at least 50 nucleotides in length but no more than about 100 nucleotides in length and comprises SEQ ID NO: 1, SEQ ID NO: 2, SEQ ID NO: 3, or SEQ ID NO: 4.

2. The guide RNA of claim 1, wherein the second nucleotide sequence is about 20-30 nucleotides in length.

3. The guide RNA of claim 1, wherein at least one nucleotide comprises a chemical 2′-modification.

4. The guide RNA of claim 1, wherein at least one linkage between the nucleotides of the guide RNA comprises a modification.

5. The guide RNA of claim 1, wherein at least one of base of the guide RNA comprises a modification.

6. The guide RNA of claim 1, wherein the guide RNA is mammalian RNA.

7. The guide RNA of claim 1, wherein the guide RNA is human RNA.

8. The guide RNA of claim 1, wherein the guide RNA binds to a nucleotide sequence of a nucleocapsid of a SARS-COV-1 and SARS-COV-2 virus.

9. The guide RNA of claim 1, wherein the guide RNA binds to a nucleotide sequence of a replicase of a SARS-COV-1 and SARS-COV-2 virus.

10. The guide RNA of claim 8, wherein the nucleotide sequence comprises RNA.

11. A pharmaceutical composition comprising the synthetic guide RNA of claim 1 and a pharmaceutically acceptable carrier.

12. The pharmaceutical composition of claim 11, further comprising a Cas13 molecule.

13. The pharmaceutical composition of claim 12, wherein the Cas13 comprises a Cas13a.

14. The pharmaceutical composition of claim 12, wherein the Cas13 molecule comprises a Cas13 protein or a Cas13 mRNA.

15. The pharmaceutical composition of claim 12, wherein the Cas13 mRNA is active in the cytosol or in the nucleus of a cell.

16. The pharmaceutical composition of claim 12, wherein the Cas13 molecule is derived from Leptotrichia buccalis, Leptotrichia wadeii, Prevotella sp. P5-125, Ruminoccocus flavefa-ciens XPD3002, Porphyromonas gulae, Ruminococcus flavefaciens, or Leptotrichia shahii.

17. The pharmaceutical composition of claim 11, wherein the pharmaceutically acceptable carrier comprises a lipid nanoparticle, a PBAE, or a PBAE-derived nanoparticle.

18. The pharmaceutical composition of claim 11, wherein the composition is formulated to deliver the guide RNA molecule and/or Cas13 molecule to the cytosol or the nucleus of the cell.

19. (canceled)

20. (canceled)

21. A method of treating or preventing a SARS-COV-1 or a SARS-CoV-2 infection in a subject, the method comprising administering a therapeutically effective amount the synthetic guide RNA of claim 1 and, a Cas13 to said subject, wherein the method decreases a SARS-COV-1 or a SARS-COV-2 viral load in the subject relative to an untreated subject with a SARS-COV-1 or a SARS-COV-2 infection.

22. A method of detecting a SARS-COV-1 or a SARS-COV-2 virus in a subject, the method comprising:
a) isolating a sample from the subject, and
b) contacting the sample with a composition comprising the synthetic guide RNA of claim 1, a Cas13, and a reporter gene, wherein the composition detects a presence of the SARS-CoV-1 or SARS-COV-2 virus.

23.-31. (canceled)


SEQUENCE LISTING (13 sequences)

SEQ ID NO: 1 - RNA, 58 nt, synthetic construct
  source 1..58: mol_type = other RNA
ggaccaccccaaaaatgaaggggactaaaacggtgtattcaaggctccctcagttgca

SEQ ID NO: 2 - RNA, 58 nt, synthetic construct
  source 1..58: mol_type = other RNA
ggaccaccccaaaaatgaaggggactaaaacgtattcaaggctccctcagttgcaacc

SEQ ID NO: 3 - RNA, 58 nt, synthetic construct
  source 1..58: mol_type = other RNA
ggaccaccccaaaaatgaaggggactaaaacctgttggtgggaatgttttgtatgcgt

SEQ ID NO: 4 - RNA, 58 nt, synthetic construct
  source 1..58: mol_type = other RNA
ggaccaccccaaaaatgaaggggactaaaacacgacatcagtactagtgcctgtgccg

SEQ ID NO: 5 - RNA, 31 nt, synthetic construct
  source 1..31: mol_type = other RNA
ggaccaccccaaaaatgaaggggactaaaac

SEQ ID NO: 6 - RNA, 38 nt, synthetic construct
  source 1..38: mol_type = other RNA
ggatttagaccaccccaaaaatgaaggggactaaaaca

SEQ ID NO: 7 - DNA, 104 bp, synthetic construct
  source 1..104: mol_type = other DNA
cgtaatgtcatccctactataactcaaatgaatcttaagtatgccattagtgcaaagaatagagctcgcaccgtagctggtgtctctatctgtagtactatgac

SEQ ID NO: 8 - DNA, 28 bp, synthetic construct
  source 1..28: mol_type = other DNA
cgtaatgtcatccctactataactcaaa

SEQ ID NO: 9 - DNA, 28 bp, synthetic construct
  source 1..28: mol_type = other DNA
aatgtcatccctactataactcaaatga

SEQ ID NO: 10 - DNA, 28 bp, synthetic construct
  source 1..28: mol_type = other DNA
gtcatccctactataactcaaatgaatc

SEQ ID NO: 11 - DNA, 28 bp, synthetic construct
  source 1..28: mol_type = other DNA
ccgtagctggtgtctctatctgtagtac

SEQ ID NO: 12 - DNA, 28 bp, synthetic construct
  source 1..28: mol_type = other DNA
tagctggtgtctctatctgtagtactat

SEQ ID NO: 13 - DNA, 28 bp, synthetic construct
  source 1..28: mol_type = other DNA
ctggtgtctctatctgtagtactatgac